(19) United States
(12) Patent Application Publication
(10) Pub. No.: US 2012/0296160 A1
(43) Pub. Date: Nov. 22, 2012

(54) Positioning Cage

(75) Inventors: Jason Hill, Brooklyn Park, MN (US); Scott R. Smith, Chaska, MN (US)

(73) Assignee: BOSTON SCIENTIFIC SCIMED, INC., Maple Grove, MN (US)

(21) Appl. No.: 13/241,961

(22) Filed: Sep. 23, 2011

Related U.S. Application Data
(60) Provisional application No. 61/487,053, filed on May 17, 2011.

(51) Int. Cl. A61B 1/00

(52) U.S. Cl. 600/104

(57) ABSTRACT

A positioning device and method of using are provided herein. The positioning device includes an expandable cage through which blood can flow. The expandable cage is insertable into a bodily organ, for example a heart, and expanded to temporarily secure the positioning device within the organ. Then, the cage can be used to steady the implantation of an annuloplasty device, for example. Alternatively, the expanded cage can be used to locate a desired area or feature of the body organ in which it is inserted by way of fluoroscopy.

### CROSS-REFERENCE TO RELATED APPLICATIONS

This application claims priority to U.S. Application No. 61/487,053, filed on May 17, 2011, the entire contents of which are hereby incorporated by reference.

### STATEMENT REGARDING FEDERALLY SPONSORED RESEARCH

Not Applicable

### FIELD OF THE INVENTION

This invention relates to a cage for positioning a prosthesis within the body. More particularly, the invention relates to a cage for positioning a prosthesis or interventional device within the heart.

### BACKGROUND OF THE INVENTION

Many situations arise in which diagnostic or interventional devices need to be positioned accurately within a patient's body. One example involves surgical procedures of the heart. More particularly, in percutaneous, endoscopic, or minimally-invasive surgical procedures of the heart, for example involving structural modification of cardiac tissue, it can be difficult to determine the exact location of the device being implanted or manipulated. In addition, it can be difficult to follow the position of the device during the surgical procedure. Further complicating the process, the device can move inside the heart as the heart beats or the patient breathes.

Maintaining a fixed location of a device relative to the heart during implantation can determine the success of the procedure. For example, mitral valve interventions, such as annuloplasty, septal repair procedures, atrial appendage interventions, pulmonary vein interventions, and other atrial wall procedures often require acute and stable positioning of diagnostic and therapeutic devices. Consequently, there remains a need for a device that can be used to stabilize, locate, and position a medical implant during and implantation procedure.

All US patents and applications and all other published documents mentioned anywhere in this application are incorporated herein by reference in their entirety.

Without limiting the scope of the invention a brief summary of some of the claimed embodiments of the invention is set forth below. Additional details of the summarized embodiments of the invention and/or additional embodiments of the invention may be found in the Detailed Description of the Invention below.

A brief abstract of the technical disclosure in the specification is provided as well only for the purposes of complying with 37 C.F.R.1.72. The abstract is not intended to be used for interpreting the scope of the claims.

### BRIEF SUMMARY OF THE INVENTION

In some embodiments, a method of implanting a medical device in heart tissue comprises the step of providing a positioning device, the positioning device comprising: a primary catheter having a lumen extending therethrough, an expandable cage, and a secondary catheter disposed through the lumen of the primary catheter. The method of implanting a medical device further comprises inserting the expandable cage into the left atrium of the heart and expanding the expandable cage to contact heart tissue and stabilize the positioning device. Further, in some embodiments, the method of implanting a medical device in the heart comprises inserting a secondary catheter through the primary catheter, the secondary catheter having the medical device attached thereto. Finally, in some embodiments, the method of implanting a medical device in heart tissue comprises attaching the medical device to a portion of the heart tissue.

These and other embodiments which characterize the invention are pointed out with particularity in the claims annexed hereto and forming a part hereof However, for a better understanding of the invention, its advantages and objectives obtained by its use, reference can be made to the drawings which form a further part hereof and the accompanying descriptive matter, in which there are illustrated and described various embodiments of the invention.

### BRIEF DESCRIPTION OF THE DRAWINGS

A detailed description of the invention is hereafter described with specific reference being made to the drawings.

FIG. 1A is a side view of positioning device in a low profile configuration.

FIG. 1B is a side view of the positioning device of FIG. 1A in an expanded configuration.

FIGS. 2A and 2B show positioning cages in expanded configurations.

FIG. 3A is a perspective view of an embodiment of a positioning device.

FIG. 3B is a cross-sectional view of the positioning device of FIG. 3A.

FIG. 4A is a perspective view of an embodiments of a positioning device.

FIG. 4B is a cross-sectional view of the positioning device of FIG. 4A.

FIGS. 5 and 6 show embodiments of the positioning cage as inserted into the atrium of a heart.

### DETAILED DESCRIPTION OF THE INVENTION

While this invention may be embodied in many different forms, there are described in detail herein specific embodiments of the invention. This description is an exemplification of the principles of the invention and is not intended to limit the invention to the particular embodiments illustrated.

For the purposes of this disclosure, like reference numerals in the figures shall refer to like features unless otherwise indicated.

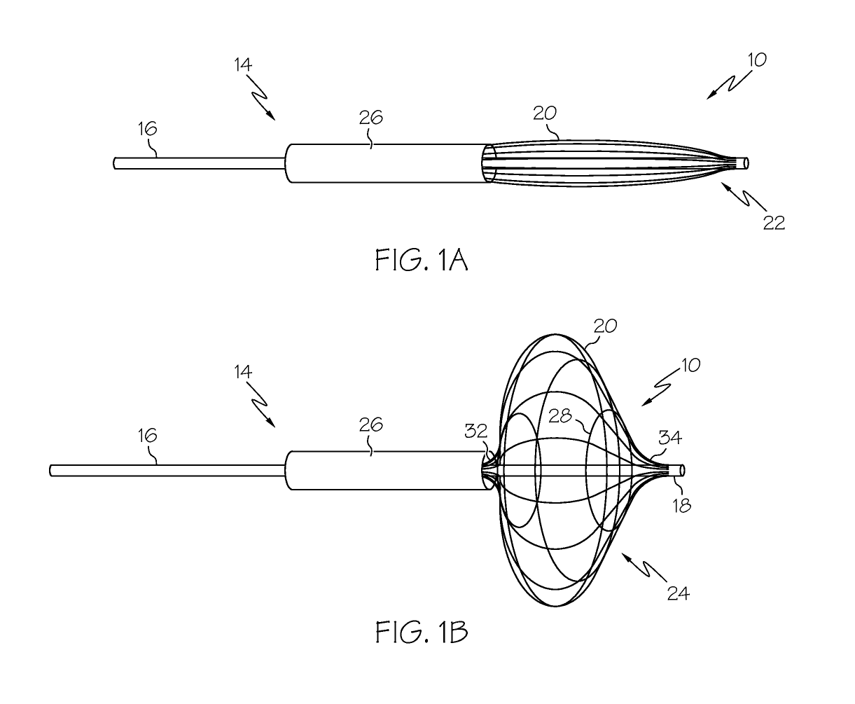

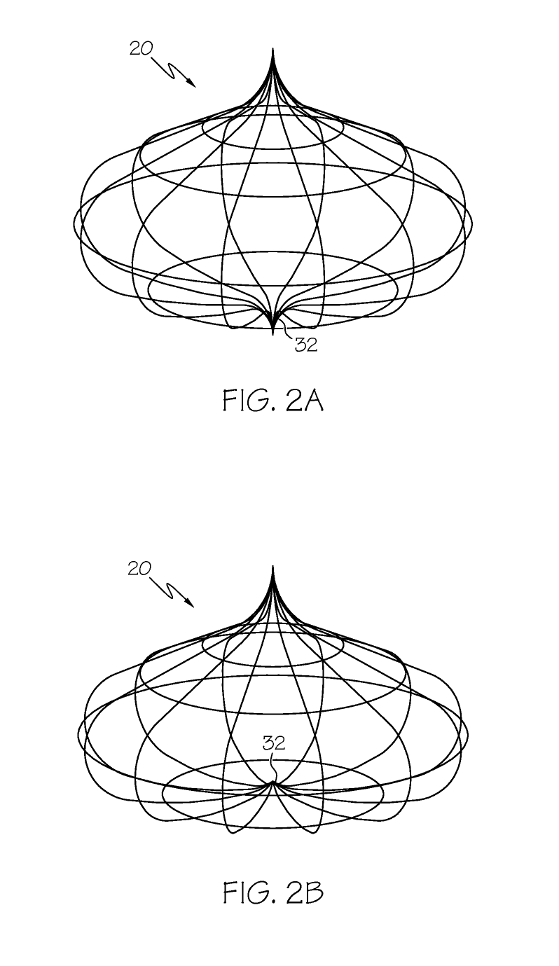

In at least one embodiment, for example as shown in FIGS. 1A, B, a positioning device 10 comprises a catheter 14 and a cage 20. In some embodiments, the positioning device 10 further comprises a control wire 16. The cage 20 comprises a low-profile configuration 22, for example as shown in FIG. 1A, and an expanded configuration 24, for example as shown in FIG. 1B. In the low-profile configuration 22, the cage 20 is insertable through a patient's vasculature. Conversely, in the expanded configuration 24, the cage 20 assumes an enlarged radial dimension.

In some embodiments, for example as shown in FIGS. 1A and 1B, the positioning device 10 further comprises a sheath 26. As further shown in FIG. 1A, the cage 20 is partially advanced out of sheath 26. With additional advancement of the cage 20 out of the sheath 26, the cage 20 expands to the expanded configuration 24.

In some embodiments, the cage 20 is attached to the control wire 16. For example, in some embodiments, the cage 20 is attached to the control wire 16 at the distal end 18 of the control wire 16. In some embodiments, the cage 20 is attached to the control wire 16 along the length of control wire 16 between the proximal end (not shown) and the distal end 18. In some embodiments, one or both ends of the cage 20 are attached to the control wire 16. Alternatively, in some embodiments, one end of the cage 20 is attached to the control wire directly and the other end is attached to a collar that slides over the control wire 16. In some embodiments, the control wire 16 is hollow, permitting a guide wire to pass through the control wire 16.

In some embodiments, the cage 20 is repositionable from a low-profile configuration 22 to an expanded configuration 24 in a reversible fashion. In this regard, the cage 20 is moveable between a low-profile configuration 22 and an expanded configuration 24 and between an expanded configuration 24 and a low-profile configuration 22. In some embodiments, the cage 20 is moved from the low-profile configuration 22 to the expanded configuration 24 by extension of the control wire 16. Extension of the control wire 16 permits the cage 20 to exit the sheath 26 and expand. In some embodiments, once the cage 20 is expanded, however, the control wire 16 can be retracted slightly so that the proximal end of the cage 20 is positioned within the sheath 26, for example as shown in FIG. 1B.

Although shown in FIGS. 1A and 1B with axial and circumferential lines, the cage 20 can comprise any suitable configuration. For example, in some embodiments, the cage 20 comprises a plurality of wires, arranged in a helical fashion, which are braided or meshed together. In some embodiments, the cage 20 comprises a braided wire structure similar to that shown U.S. Pat. No. 5,061,275 to Wallsten et al., which is herein incorporated by reference. In some embodiments, the wires of the cage 20 comprise a super-elastic shape memory material, for example a nickel-titanium alloy such as Nitinol®. The wires can also comprise a stainless steel alloy, a nickel-cobalt-chromium-molybdenum alloy such as MP35N®, a predominantly nickel-chromium alloy such as Inconel®, or cobalt-chromium-nickel alloy such as Elgiloy®. In some embodiments, the cage 20 comprises a non-metallic material, or materials, in lieu of or in addition to metallic wires.

In some embodiments, the cage 20 has a self-deploying tendency, for example by employing elastic and/or thermal memory properties. In some embodiments, the cage 20 comprises a slotted-tube expandable structure. In some embodiments, blood or other fluid is permitted to flow freely through the cage 20, for example where the cage 20 is disposed inside the heart.

In some embodiments, the cage 20 comprises a wire mesh 28 that has a uniform density throughout the cage 20. In some embodiments, however, the density of the wire mesh 28 varies with the region of the cage 20. For example, in some embodiments, the wire mesh 28 is more dense at the distal end 34 of the cage 20 than at the proximal end 32 of the cage 20. Alternatively, in some embodiments, the wire mesh 28 is more dense at the proximal end 32 of the cage 20 than at the distal end 34 of the cage 20. Other density configurations are also suitable. In some embodiments, the density of the wire mesh 28 varies by an increase or decrease in the number of wires.

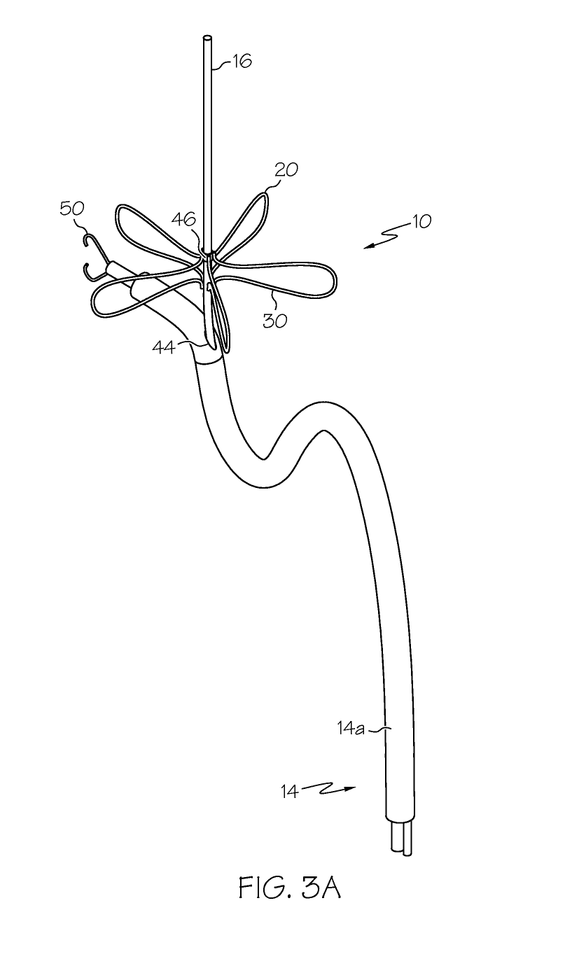

Turning to FIG. 2A, the cage 20 is shown therein having a bulbous shape. In some embodiments, the cage 20 has a first end 32 that is convex, when viewed from outside the cage 20. Alternatively, in some embodiments, for example as shown in FIG. 2B, the cage 20 has a first end 32 which is concave, when viewed from outside the cage 20. A concave first end 32 is employed, in some embodiments, in order to keep from interfering with the mitral valve. In addition, the first end 32 of the cage 20 can serve as either of the distal or proximal end of the cage, depending upon the deployment route. For example, the orientation of the cage 20 on the catheter 14 can depend on whether the catheter 14 is inserted into a heart cavity, for example, via a retrograde approach or antegrade approach.

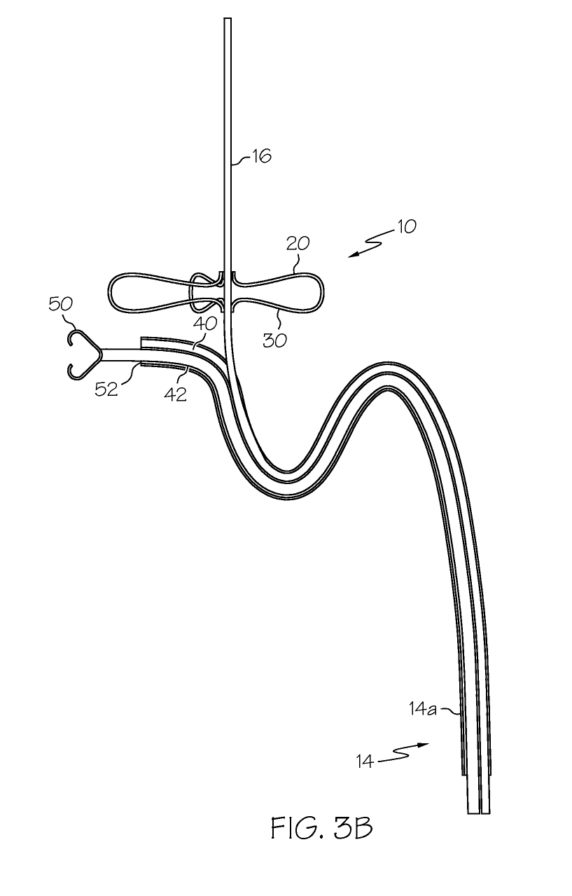

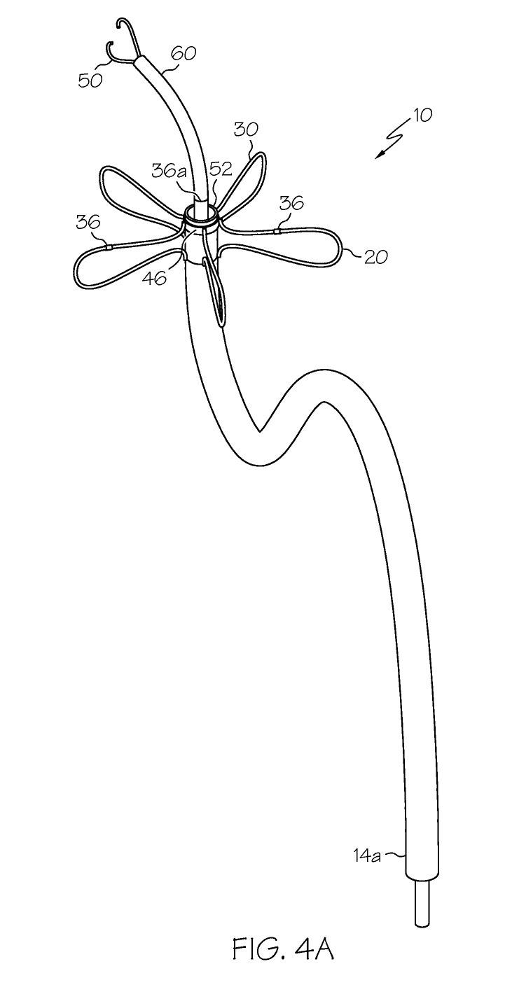

With regard to FIGS. 3A and 3B, in some embodiments, the catheter 14 of the positioning device 10 comprises a primary catheter 14a. As shown in FIGS. 3A and 3B, the primary catheter 14a includes plurality of lumens, for example a first lumen 40 and a second lumen 42. In some embodiments, the cage 20 and control wire 16 are routed through the first lumen 40, while a medical device 50 is routed through the second lumen 42. In some embodiments, the control wire 16 exits the primary catheter 14a at a different location than the medical device 50. For example, in some embodiments, the medical device 50 exits the primary catheter 14a at the distal end 52 thereof. And, in some embodiments, the control wire 16 exits the primary catheter 14a at an opening 44 that is proximal to the distal end 52.

In some embodiments, for example as shown in FIGS. 3A and 3B, the cage 20 is attached to the control wire 16 with a sildable collar 46. The slidable collar 46 moves axially along a portion of the control wire 16 to expand or contract the cage 20 as desired. In some embodiments, the wires 30 of the cage 20 are secured at one end to the slidable collar 46, while the other end of the wires 30 is secured directly to the control wire 16. In some embodiments, the catheter 14 comprises a steerable section that is distal to the exit of the control wire 16.

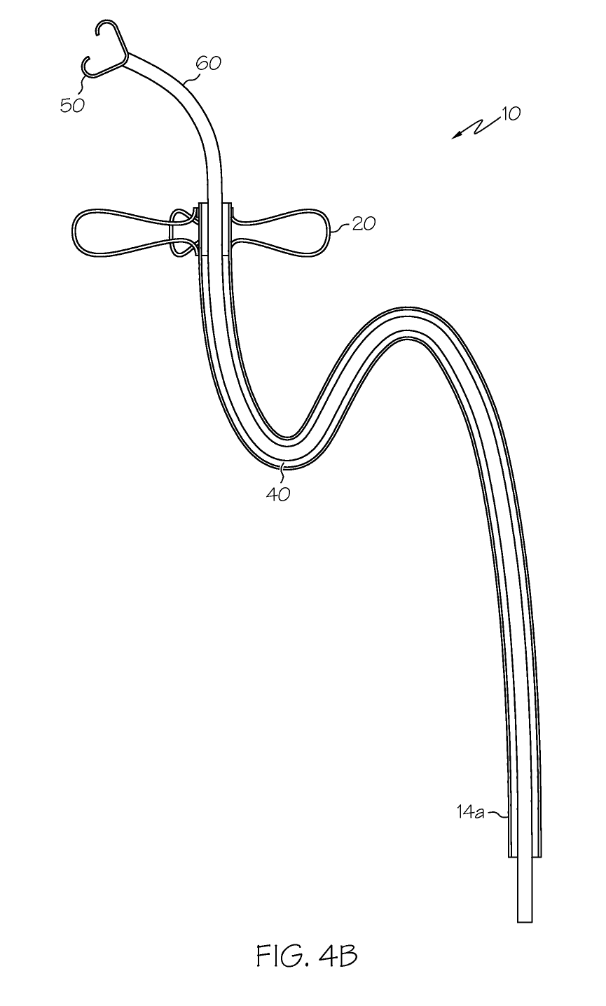

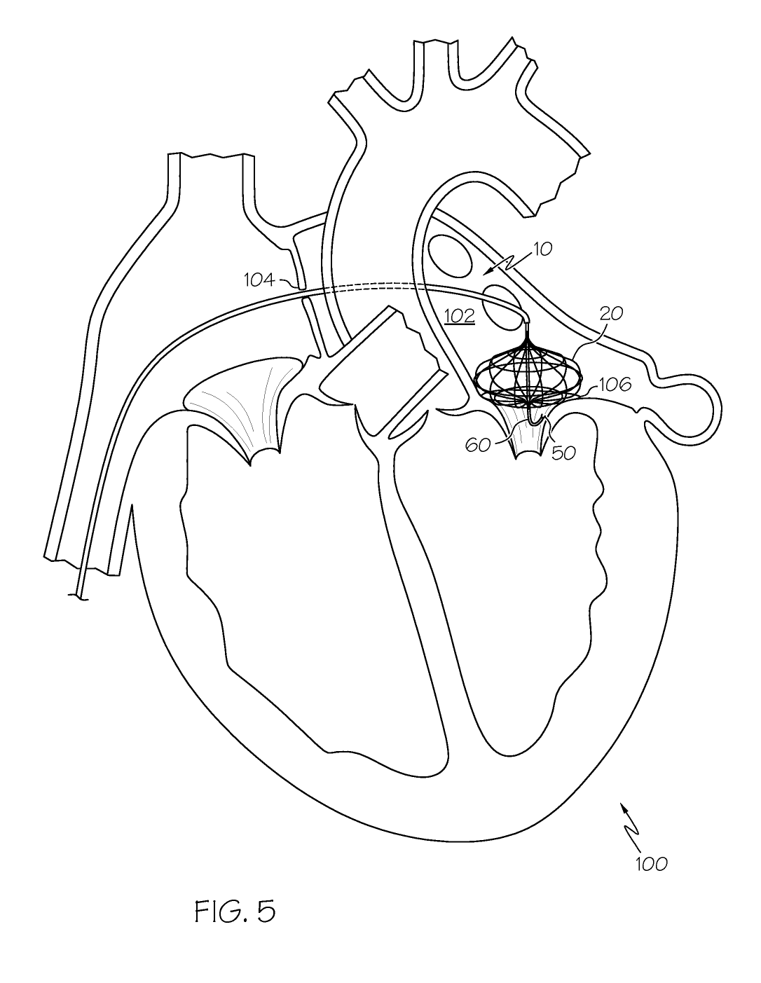

Turning to FIGS. 4A and 4B, in some embodiments, a positioning device 10 comprises a secondary catheter 60 which is extendable from the distal end 52 of the primary catheter 14a. The secondary catheter 60 has a medical device 50 attached thereto. As shown in FIGS. 4A and 4B, the cage 20 is attached to the primary catheter 14a. In some embodiments, the cage 20 is attached to a slidable collar 46 that is sildable with respect to the primary catheter 14a.

With further regard to FIGS. 4A and 4B, the secondary catheter 60 and the primary catheter 14a are concentric along at least a portion of the length of the primary catheter 14a. In some embodiments, the secondary catheter 60 and primary catheter 14a are concentric along the entire length of the primary catheter 14a.

Turning specifically to FIG. 4B, a cross-sectional view of the positioning device 10 is shown therein. In some embodiments, the positioning device 10 comprises a single or first lumen 40 through which the secondary catheter 60 is routed. Although shown with the secondary catheter 60 extending therethrough, additional devices can also be routed through the first lumen 40.

In some embodiments, for example in accordance with FIGS. 3A, 3B, 4A, and 4B, the positioning device 10 is used in combination with a medical device 50 which is manipulated after the positioning cage 20 is expanded within a body cavity, for example the heart. In some embodiments, the medical device 50 comprises a device as disclosed in one or more of the following Applications: “Percutaneous Mitral Annulus Mini-Plication,” with Application No. 61/487,065; “Corkscrew Annuloplasty Device,” with Application No. 61/487,063; “Annuloplasty Ring with Piercing Wire and Segmented Wire Lumen,” with Application No. 61/487,072; and “Annuloplasty Ring with Anchors Fixed by Curing Polymer,” with Application No. 61/487,083, all of which were filed on May 17, 2011, the contents of which are hereby incorporated by reference.

In some embodiments, at least one end of the cage 20 is partially open to permit the secondary catheter 60 and/or medical device 50 to pass therethrough. Alternatively, in some embodiments, both ends of the cage 20 are closed.

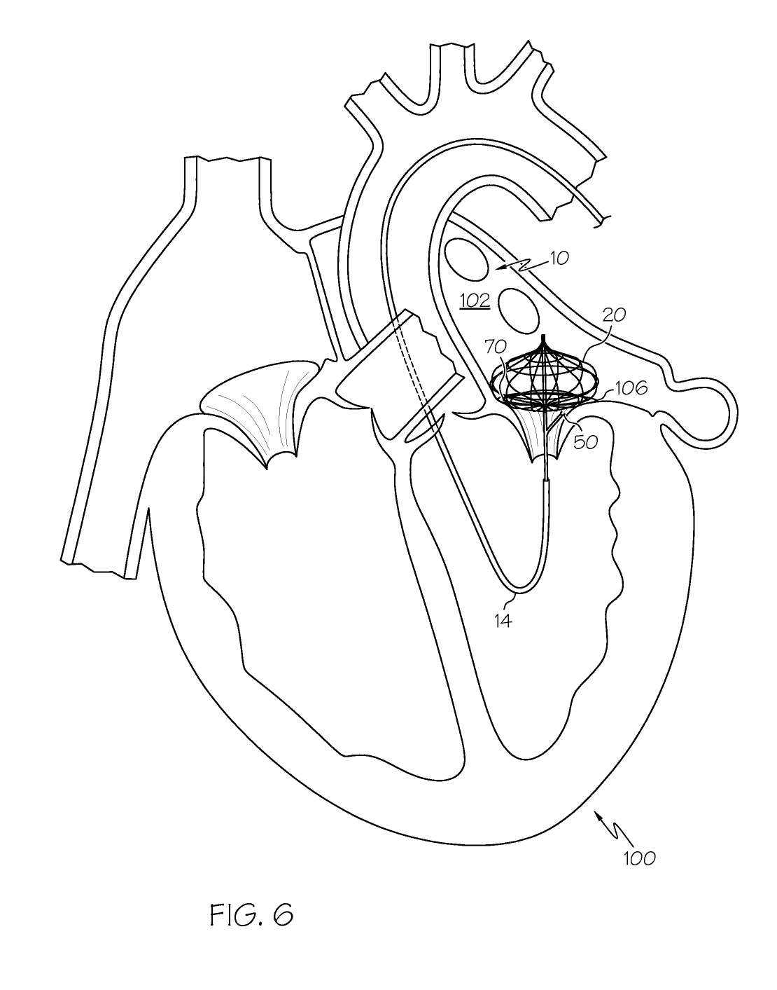

Turning now to FIG. 5, in some embodiments, the positioning device 10 is introduced into the left atrium 102 of the heart 100 through the atrial septum 104. In particular, in some embodiments, the positioning device 10 is introduced percutaneously in the low-profile configuration 22 via an antegrade approach, through the foramen ovale. Other minimally-invasive approaches are also suitable, for example as described in greater detail below. Subsequently, the cage 20 is deployed, for example, at the mitral annulus 106.

In some embodiments, upon introduction into the left atrium 102, the positioning device 10 is expanded to contact the endocardium. Upon expansion to the expanded configuration 24, in some embodiments, contact with the endocardium stabilizes the cage 20 within the left atrium 102. After the cage 20 is expanded and stabilized, in some embodiments, the medical device 50 is deployed. As shown in FIG. 5, for example, a secondary catheter 60 is deployed through the primary catheter 14a and cage 20. In some embodiments, the secondary catheter 60 is moveable relative to the cage 20. In particular, in some embodiments, the secondary catheter 60 is used to carry out an annuloplasty procedure. In this way, the cage 20 is used in combination with the medical device 50 to carry out an annuloplasty procedure. Finally, the cage 20 can also be used to carry out treatment of the mitral valve, atrial structure occlusion, ventricular structure repair, electrical monitoring or cardiac rhythm therapy, or other cardiac valve treatment.

In some embodiments, the cage 20 is comprised of a plurality of wires 30 forming loops or leaflets. In addition, in some embodiments, the wires 30 are joined or meshed to form a diamond or hexagonal pattern. Further, the cage 20 can be formed from a slotted tube material, a single wire bent to intersect itself, or a plurality of non-intersecting wires shaped into a cone or bell. Finally, in some embodiments, the wires of the plurality of non-intersecting wires are connected by polymer fibers or yarn to stabilize the wires and maintain their position.

In some embodiments, the secondary catheter 60 is steerable. Moreover, in some embodiments, the cage 20, upon expansion, provides an unobstructed area within which the secondary catheter 60 can be manipulated, for example, to permit the placement of a medical device 50 within adjacent tissue. Also, in some embodiments, the cage 20 has large enough openings to permit the secondary catheter 60 to move in and out of the cage 20, for example, to access the mitral annulus 106. In some embodiments, the positioning device 10 is introduced via a trans-apical approach, for example with a combination catheter.

Turning now to FIG. 6, in some embodiments, the positioning device 10 is introduced into the left atrium 102 of the heart 100 by way of a retrograde arterial access route. In this way, in some embodiments, the cage 20 is used to snug an annuloplasty ring 70 or other device against the mitral annulus 106. This can be accomplished by introducing the cage 20 into the left atrium, as shown in FIG. 6, and subsequently pulling on the catheter 14, thereby applying pressure on an annuloplasty ring 70 abutting the mitral annulus 106. Furthermore, an annuloplasty ring 70 can be introduced prior to expansion of the cage 20. In some embodiments, the annuloplasty ring 70 is introduced via a transseptal approach, positioned at or near the mitral annulus 106, and then the cage 20 is expanded to press the annuloplasty device against the mitral annulus 106. Subsequently, the annuloplasty device 70 is secured to the mitral annulus 106 as desired.

In addition, in some embodiments, the positioning device 10 shown in FIG. 6 is used in combination with the medical device 50. In some embodiments, the medical device 50 is disposed proximally to the cage 20, for example as shown in FIG. 6. In this way, in some embodiments, the medical device 50 is used to carry out treatment of the mitral valve or annulus. In some embodiments, however, the positioning device 10 does not include medical device 50. Also, in some embodiments, the annuloplasty ring 70 or other medical device is inserted via a catheter that is wholly separate from catheter 14, for example, where the annuloplasty ring 70 or other device is introduced transvenously.

In some embodiments, the cage 20 is radiopaque or at least partially radiopaque. In particular, in some embodiments, the wires 30 of the cage 20 comprise radiopaque material. In some embodiments, some of the wires 30 of the cage 20 comprise radiopaque material. Alternatively, or in addition, sections of the wires 30 are radiopaque. In some embodiments, the cage 20 is rendered radiopaque via radiopaque markers that are added to the cage 20 or wires 30 to aid in visualization of the cage 20, or specific portions of the cage 20, during the procedure.

In some embodiments, the cage 20 serves as an imaging indicator to ensure the operator can locate the mitral valve. In addition, in some embodiments, the cage 20 can be used to make other body cavities or features visible by differential contrast or radiopacity. For example, the cage 20 is used to provide visibility for various points along the atrial wall, the atrial appendage, pulmonary veins, and septal wall. Also, the positioning device 10 can be used to conduct valve repair or diagnostic procedures to coronary or arch vessels, for example in the aortic sinus or aortic arch. Returning to FIG. 4A, in some embodiments, the cage 20 has a plurality of radiopaque markers 36 thereon to facilitate accurate placement of cage 20 within the heart or other bodily cavity. In addition, in some embodiments, the secondary catheter 60 has at least one radiopaque marker 36a thereon to aid in locating and guiding the secondary catheter 60. The at least one radiopaque marker 36a on the secondary catheter 60 is located in any desirable location or locations, including immediately adjacent to the distal end of the secondary catheter or along the length of the secondary catheter 60.

In addition to or in lieu of radiopaque markers 36, 36a, some embodiments comprise markings or position sensors to aid in guiding the cage 20, secondary catheter 60, and/or primary catheter 14a. Further, in some embodiments, radiopaque markers 36, 36a, position sensors, or other markings on the secondary catheter 60 indicate rotation of the secondary catheter 60 with respect to the cage 20. The cage 20 can also include asymmetrical markings, for example single markers on some of the wires 30 and double markers on other wires, to angiographically distinguish near and far portions of the cage 20. This allows for orientation of the cage 20. Also, radiopaque markers 36 on the cage 20 can be used as circumferential targets for proper placement of the cage 20 within the heart or body cavity.

In some embodiments, the cage 20 is attached to a slidable collar 46 that is attached to the end of a tube. The tube is internally concentric with the primary catheter 14a. When the cage 20 is fully expanded, the slidable collar 46 is in contact with the distal end 52 of the primary catheter 14a.

Alternatively, in some embodiments, the wires 30 can be advanced and withdrawn within the primary catheter 14a to respectively expand and contract the cage 20.

In addition to the foregoing, in some embodiments, the positioning device 10 is introduced via a minimally-invasive thoracic access, for example, lateral thoracotomy or apical access. Also, in some embodiments, the positioning device 10 is configured to follow a guidewire. And, in some embodiments, the guidewire is exchanged for the cage 20. Alternatively, in some embodiments, both a guidewire and cage 20 can be utilized simultaneously.

In some embodiments, the positioning device 10 comprises a plurality of lumens, for example, a guidewire lumen, inflation lumen for inflating a balloon, contrast media lumen for injecting contrast media, medication lumen for injecting medication, suction lumen. The positioning device 10 can further comprise various combinations of the foregoing lumens. In addition, certain lumens can be dual purpose lumens, carrying out one or more of the functions described above.

The positioning device 10 is not limited to use with or in the heart. In some embodiments, it is used for stone extraction, positioning of tumor ablation catheters or prostate treatment devices. Also, in some embodiments, the positioning device 10 comprises a plurality of cages 20, which can further improve the accuracy of deployment, improving positioning. In particular, in some embodiments, the cage 20 is temporarily anchored within the heart such that when the heart beats, the cage 20 moves with the heart. The cage 20 thereby maintains a fixed position relative to the heart. Also, the cage 20 can be fixed with respect to the mitral annulus 106. This, in turn, allows for greater accuracy in placing a medical device or annuloplasty ring, while reducing risk to the patient.

In some embodiments, the cage 20 has a covering over a portion or the entirety of the cage 20. The covering can be on the outside surface of the cage 20, the inside surface, or both. In some embodiments, the covering limits or prevents fluid passage through the cage, or a portion thereof. Consequently, in some embodiments, the covering is used to prevent blood flow, reduce passage of contrast media, medication, or gaseous material.

In some embodiments, the secondary catheter 60 exits the primary catheter 14a proximal to the cage 20 and the secondary catheter 60 is steerable.

In addition, the device is not limited to use in the heart. It can also be placed in other body organs, for example, the lungs, bladder, stomach, or intestine.

In some embodiments, a method of implanting a medical device in heart tissue comprises providing a positioning device 10. The positioning device 10 comprises a primary catheter 14 having a lumen 42 extending therethrough. The positioning device 10 further comprises an expandable cage 20 comprising a braided wire mesh. And, the positioning device 10 further comprises a secondary catheter 14a disposed through the lumen of the primary catheter 14a. The method further comprises inserting the expandable cage 20 into the left atrium of the heart, expanding the expandable cage 20 to contact heart tissue and stabilize the positioning device 10. In addition, in some embodiments, the method comprises inserting a secondary catheter 60 through the primary catheter 14a, the secondary catheter 60 having a medical device 50 attached thereto. The method further comprises attaching the medical device 50 to a portion of the heart tissue.

In some embodiments, the method of implanting a medical device further comprises attaching the medical device 50 to the mitral annulus.

In some embodiments, the method further comprises deploying the cage 20 from a sheath 26.

In some embodiments, a method of positioning an annuloplasty device along at least a portion of the mitral annulus comprises providing a catheter 14 comprising a sheath 26 and an expandable positioning cage 20, inserting the catheter 14 and expandable positioning cage 20 in the atrium of a heart, inserting the annuloplasty device in to the atrium, deploying the positioning cage 20, and pushing the annulpplasty device against the mitral annulus with the positioning cage 20.

In some embodiments, the positioning cage 20 is self expanding. In some embodiments, the method of positioning an annuloplasty device further comprises deploying the positioning cage via a control wire 16.

The above disclosure is intended to be illustrative and not exhaustive. This description will suggest many variations and alternatives to one of ordinary skill in this field of art. All these alternatives and variations are intended to be included within the scope of the claims where the term “comprising” means “including, but not limited to.” Those familiar with the art may recognize other equivalents to the specific embodiments described herein which equivalents are also intended to be encompassed by the claims.

Further, the particular features presented in the dependent claims can be combined with each other in other manners within the scope of the invention such that the invention should be recognized as also specifically directed to other embodiments having any other possible combination of the features of the dependent claims. For instance, for purposes of claim publication, any dependent claim which follows should be taken as alternatively written in a multiple dependent form from all prior claims which possess all antecedents referenced in such dependent claim if such multiple dependent format is an accepted format within the jurisdiction (e.g. each claim depending directly from claim 1 should be alternatively taken as depending from all previous claims). In jurisdictions where multiple dependent claim formats are restricted, the following dependent claims should each be also taken as alternatively written in each singly dependent claim format which creates a dependency from a prior antecedent-possessing claim other than the specific claim listed in such dependent claim below.

This completes the description of the preferred and alternate embodiments of the invention. Those skilled in the art may recognize other equivalents to the specific embodiment described herein which equivalents are intended to be encompassed by the claims attached hereto.

What is claimed is:

1. A method of implanting a medical device at the mitral valve of a heart, the method comprising:
providing a positioning device comprising:
a primary catheter having a lumen extending therethrough;
an expandable cage comprising a braided wire mesh; and
a secondary catheter disposed through the lumen of the primary catheter;
inserting the expandable cage into the left atrium of the heart;
expanding the expandable cage into a bulbous configuration, the expandable cage thereby contacting heart tissue and stabilizing the positioning device;
inserting a secondary catheter through the primary catheter, the secondary catheter having the medical device attached thereto; and
attaching the medical device to a portion of the heart tissue adjacent the mitral valve.

2. The method of claim 1, wherein the step of attaching the medical device to a portion of the heart tissue comprises attaching the medical device to the mitral annulus.

3. The method of claim 1, wherein the expandable cage is at least partially radiopaque.

4. The method of claim 1, wherein the positioning device further comprises a sheath, the sheath disposed over at least a portion of the expandable cage prior to expansion of the expandable cage.

5. The method of claim 4, wherein the step of inserting the expandable cage into the left atrium of the heart comprises inserting the primary catheter through the atrial septum.

6. The method of claim 5, wherein the step of expanding the expandable cage further comprises pushing the expandable cage out of the sheath.

7. A method of positioning an annuloplasty device along at least a portion of the mitral annulus within the atrium of a heart, the method comprising:
providing a catheter comprising a sheath and an expandable positioning cage;
inserting the catheter and expandable positioning cage into the atrium;
inserting the annuloplasty device into the atrium;
deploying the positioning cage such that upon deployment, the positioning cage takes on a bulbous configuration; and
pushing the annuloplasty device against the mitral annulus with the positioning cage.

8. The method of claim 7, wherein the positioning cage is self-expanding.

9. The method of claim 7, wherein the step of deploying the positioning cage comprises extending the positioning cage from the sheath.

10. The method of claim 7 further comprising providing a control wire attached to the positioning cage.

11. The method of claim 10, wherein the step of deploying the positioning cage comprises pushing the positioning cage out of the sheath with the control wire.
